(19) United States
(12) Patent Application Publication
(10) Pub. No.: US 2017/0000940 A1
(43) Pub. Date: Jan. 5, 2017

(54) METHOD FOR ESTABLISHING AND/OR MONITORING THE STATE OF AN EXTRACORPOREAL FLUID OR FLUID FLOW BY MEANS OF ULTRASOUND

(71) Applicant: GAMPT MBH, GESELLSCHAFT FÜR ANGEWANDTE MEDIZINISCHE PHYSIK UND TECHNIK, Merseburg (DE)

(72) Inventors: Michael Schultz, Bad Duerrenberg (DE); Robert Klaua, Halle (Saale) (DE); Georg Dietrich, Salzmuende OT Zappendorf (DE); Anton Schlesinger, Naumburg (DE)

(73) Assignee: GAMPT mgH Gesellschaft fur Angewandte Medizinische Physik und Technik, Merseburg (DE)

(21) Appl. No.: 15/039,992

(22) PCT Filed: Nov. 21, 2014
(86) PCT No.: PCT/EP2014/075304

§ 371 Date: May 27, 2016

(30) Foreign Application Priority Data
Nov. 28, 2013 (DE) 10 2013 224 389.8

(51) Int. Cl. A61M 1/36; G01N 29/024; G01N 33/49; G01N 11/02

(52) U.S. Cl. CPC A61M 1/3663; A61M 1/367; A61M 1/3672; G01N 11/02; G01N 29/024; G01N 33/4905; A61M 2205/3334; A61M 2205/3375; A61M 2205/50; G01N 2011/0073

(57) ABSTRACT

The invention relates to a method for establishing and/or monitoring foreign structures in an extracorporeal fluid or in a fluid flow, in particular in blood or a bloodstream, wherein the fluid is monitored by means of ultrasound. The method according to the invention is characterized in that the features of the fluid state established by means of the ultrasound monitoring are processed by means of a multi-criteria ultrasonic analysis. The invention furthermore relates to a device for performing this method and the use of this device.

### DESCRIPTION

The invention relates to a method for establishing and/or monitoring the state of an extracorporeal fluid or fluid flow, in particular of blood or a bloodstream, wherein the fluid is monitored by means of ultrasound. The method according to the invention is characterized in that the features of the fluid state established by means of the ultrasonic monitoring are processed with a multi-criteria ultrasonic analysis. The invention furthermore relates to a device for carrying out this method and to the use of this device.

A distinction is made among various extracorporeal methods. The most widely used is hemodialysis, and use is also made of hemofiltration and hemodiafiltration. Other extracorporeal methods include hemoperfusion, which is used for certain forms of acute poisoning, and apheresis. Extracorporeal blood circulatory systems (extracorporeal circuits) are also used in heart-lung machines.

By hemodialysis is meant the removal of fluid and dissolved molecules from the extracorporeally circulating blood via filter systems, which generally contain a semipermeable membrane. Hemodialysis is a so-called kidney replacement method. Along with kidney transplanting, dialysis is the most important kidney replacement therapy for chronic kidney failure and one of the treatment options for acute kidney failure.

Therapeutic apheresis, also known as blood cleaning or blood scrubbing, is a method for the extracorporeal removal of pathogenic constituents such as proteins, protein-bound substances, and cells from the blood or blood plasma of patients. The cleansed blood is returned [to the patient's body] after the pathogenic substances have been removed. Apheresis can also be used to obtain blood constituents from an individual for use as, for example, donor substances. Apheresis methods are used in particular for obtaining sufficient amounts of blood constituents from individual donors that only make up a small percentage of the blood, such as thrombocytes or blood stem cells. In apheresis methods, the donor's blood is drawn from the arm vein and conducted into a closed, sterile, single-use only tube system. There it is mixed with the necessary amount of anticoagulants to prevent the blood from clotting in the apheresis system. This mixture is conducted into a centrifuge, in which the blood constituents are separated into layers according to their density. The desired blood constituents can now be separated out. All of the unneeded blood constituents are returned to the donor's body.

Apheresis is used in, for example, modern cancer therapy or for treating various blood diseases, for example for the disease Polycythaemia vera.

The heart-lung machine is a medical device that can replace the pumping function of the heart and also pulmonary function for a limited period of time. In this device, the blood undergoes extracorporeal circulation, wherein it is drawn from the body via a tube system, enriched with oxygen, and returned to the body. The heart-lung machine is most commonly used in heart surgery; smaller (so-called extracorporeal membrane oxygenation, ECMO) systems are used in emergency and intensive medicine. Microembolisms are a known problem associated with the use of heart-lung machines. Microembolisms can be caused by fibrin clots or by plastic particles abraded from the tube surfaces or coming from the oxygenator of the heart-lung machine.

Although the blood is treated with anticoagulants (for example heparin, which is the most commonly used anticoagulant), there is still the risk of blood clots occurring. There are various causes for this. Along with incorrect dosage of anticoagulants, temporary blood stasis can occur in the extracorporeal circuit. Owing to the design specifications of the medical devices employed, in some circumstances stagnations may arise. Contact with air or with the plastic surfaces of the extracorporeal blood circulatory system can also trigger blood clotting.

The blood's ability to clot (coagulate) is necessary in order to stop bleeding in the event of an injury, for example. Hence it is one of the most important properties of blood. The thrombocytes (platelets) play an extraordinarily important role in blood clotting. Blood clotting follows the so-called clotting cascade. After they are activated, the thrombocytes and other coagulation proteins contained in the blood begin to aggregate and form a thrombus (also known as a clot). The rate of clot formation depends upon the amount of active coagulation proteins.

Although natural and essential to life, in certain cases blood clotting can be harmful, specifically when blood coagulates in the cardiovascular system. Equally problematic is blood coagulating in the extracorporeal circuit of, for example, a hemodialysis machine, a heart-lung machine, or an apheresis apparatus. This can result in the formation of a blood clot (thrombus) in the extracorporeal circuit. The thrombus can obstruct thin capillaries in the extracorporeal circuit. There is also the risk of a thrombus formed in the extracorporeal circuit reaching the patient's circulatory system and obstructing a blood vessel therein to such a degree that blood no longer flows in the subsequent supply area and thus the oxygen and nutrient supply is interrupted (thrombosis). This can lead to tissue death and even to the partial failure of certain organs.

In the extracorporeal blood circuit of, for example, a hemodialysis machine, a heart-lung machine, or an apheresis apparatus, it is thus essential to monitor the coagulation state of the blood during the treatment and changes in the coagulation state that may arise during the treatment or that may be triggered by the treatment. In particular this includes the monitoring of changes in the distribution of blood constituents, in particular of blood cells, and the monitoring of changes in blood viscosity. The purpose of this is early detection of undesired incipient blood coagulation and the possible formation of blood clots associated therewith. Changes in the coagulation state of the blood, blood clots, and other hazardous constituents need to be corrected and/or retained in the extracorporeal circulatory system by means of suitable measures in order to avoid adverse impacts on the patient undergoing treatment.

Standard treatment systems therefore comprise means for detecting hazardous constituents (e.g., air bubbles) in the extracorporeal blood circulatory system and furthermore have suitable mechanisms for triggering an alarm and/or stopping the treatment. Use is made of so-called clot catchers in an attempt to retain blood clots before they can reach the patient's body.

Methods and devices for monitoring extracorporeal circuits for hazardous constituents contained therein are known from the prior art.

DE 10 2010 034 553 discloses a device for the detection and/or monitoring of foreign structures in a fluid or fluid flow as well as a method for doing so. This system is in particular capable of detecting air bubbles in the fluid flow by means of an ultrasonic monitoring means. However, blood clots cannot be detected in the blood by means of ultrasound, but only with an additional optical monitoring means.

DE 2911258 B1 discloses a device for the non-invasive measurement of the blood flow rate according to the ultrasonic Doppler Effect method, with which the blood flow rate in the area of the small and smallest vessels (microcirculation) can be measured and/or an erythrocyte aggregation can be detected.

US 5928180 discloses a method and a device for real time monitoring of the blood volume in a blood filter, which make it possible to monitor the filter and the performance of a dialysis machine and to give timely warning about an imminent coagulation .

DE 10311408 B3 discloses a method for the non-invasive measurement of the concentration of blood constituents in central blood vessels, in particular the hemoglobin concentration or oxygen saturation of the blood, by measuring light backscattered under the effect of ultrasonic irradiation.

However, thus far it has not been possible to detect changes in the coagulation state, in particular the onset of undesired blood clotting, in an instantaneous and reliable manner with the methods and devices disclosed in the prior art. The methods and devices based on the scattering effect of an ultrasonic signal on blood clots are not reliable, because the scattering effect of blood clots is generally too small.

It is thus the object of the present invention to provide a method and a device that make it possible to detect changes in the state of blood, in particular in the clotting state, quickly, safely, and with the simplest possible measuring set-up. The object of the invention is in particular the detection of characteristics of human blood that deviate from the normal state and the monitoring of the temporal progression of changes in the blood state that lead to deviations from the normal state.

The mean values of exemplary physical blood characteristics for a healthy adult human are given in Table 1.

TABLE 1
Average characteristics of human blood

| | |
|---|---|
| Color | Red |
| Aggregation state | Fluid |
| Temperature range considered | 35.8° C. to 37.2° C. |
| Density | 1.057 g/ml |
| Viscosity at 37° C. | 4 mPa · s to 25 mPa · s |
| Sonic velocity | 1483 m/sec |
| Erythrocyte diameter | 6 μm to 15 μm |
| Hematocrit | 37% to 50% |

The method according to the invention and the device according to the invention should make it possible to detect the occurrence of undesired changes in the blood state, in particular undesired clotting instantaneously in an extracorporeal circuit as early as possible and with high reliability.

This object is achieved by a method with the features of claim 1. Provision is made of a method for establishing and/or monitoring the state of a fluid or of a fluid flow, wherein the fluid is monitored by means of ultrasound. The features of the fluid state established by means of the ultrasonic monitoring are then processed with a multi-criteria ultrasonic analysis, in particular with an analysis algorithm, by which a change in the distribution of particles contained in the fluid and/or changes in the viscosity of the fluid are measured and the fluid state is assigned to previously defined states.

The fluid or fluid flow is preferably a liquid that can contain dissolved substances as well as suspended particles. In a preferred embodiment of the invention, the fluid is a suspension. A suspension is a heterogeneous mixture of a liquid and solid particles finely dispersed therein, which are slurrified and held in suspension in the liquid by means of suitable units (stirrers, dissolvers, liquid jets, wet mills) and usually also with additional dispersal agents. A suspension is a coarsely dispersed dispersion with a tendency to sedimentation and phase separation. The solid substances are suspended in the liquid phase. The fluid state is in particular the mixed or separation state of the individual phases.

In another preferred embodiment of the invention, the fluid is a dispersion. A dispersion is a heterogeneous mixture of at least two substances that do not or barely dissolve in each other or chemically combine with each other. In such a fluid, a substance (dispersed phase) is finely dispersed in another substance (dispersion medium). As a rule they are colloids. The individual phases can be clearly distinguished from one another and as a rule separated from one another by physical methods (e.g., filtering, centrifuging), or else they separate on their own (sediment). The fluid state is in particular the mixed or separation state of the individual phases.

In another preferred embodiment of the invention, the fluid is an emulsion. By an emulsion is meant a finely dispersed mixture of two normally immiscible liquids, without visible separation. Examples of emulsions are numerous cosmetics, milk, or mayonnaise. The fluid state is in particular the mixed or separation state of the individual liquids.

In a particularly preferred embodiment of the invention, the fluid is blood, most preferably blood in a circulatory system such as an extracorporeal circuit. The fluid state is in particular the coagulation state of blood.

In a preferred embodiment, provision is made of a method for establishing and/or monitoring the state of blood or of a bloodstream, in particular of an extracorporeal bloodstream, wherein the blood is monitored by means of ultrasound. The features of the blood state established by means of the ultrasonic monitoring are processed with a multi-criteria ultrasonic analysis, in particular with an analysis algorithm, by which a change in the distribution of constituents in the blood and/or changes in the viscosity of the blood are measured and the blood state is assigned to previously defined states, in particular to clotting states.

The method according to the invention has the advantage that changes in the state of a fluid, in particular in the clotting state of blood, can be established early, preferably from the beginning of the onset of the state change on.

With the method according to the invention, it is also possible to detect at least one foreign structure or distinguish it from at least one second foreign structure in the fluid.

The at least one first foreign structure is in particular a solid body, preferably a blood clot.

The at least one second foreign structure can be another solid body, for example a foreign body detached from a surface of the extracorporeal circuit, such as abrasion products from the fluid conduit means. However, the one second foreign structure can also be an air bubble, for example.

With the method according to the invention, it is preferably possible to distinguish between blood clots and other kinds of foreign bodies such as air. The differentiation can be made using the backscattering amplitude, which for example is greater for air bubbles than for blood clots because the impedance jump between blood and air is increased.

Any standard prior art measuring method can be used for the ultrasonic monitoring. Preference is given to performing the ultrasonic monitoring by measuring backscattering, frequency shifting (Doppler), and/or viscosity (elastography).

The method is based in particular on the amplification of the ultrasonic backscattering signal when blood clots occur. Blood coagulation (hemostasis) is a complex biochemical process, and neither the progression of the process nor the substances involved in it have been fully investigated. When coagulation is triggered, one or more clotting factors are activated, which in turn activate other factors in a cascade-like fashion.

From a physical chemistry standpoint, blood is a suspension of blood cells in blood plasma. There are three main types of cells: red corpuscles (erythrocytes), white corpuscles (leukocytes), and platelets (thrombocytes). As can be seen in Table 2, the scattering cross section, which is defined as the product of the area and the number, is much greater for red corpuscles than for any other type of cell.

TABLE 2
Comparison of blood cells

| | Diameter | | Total area π/4(d²N) |
|---|---|---|---|
| Type | d [μm] | No. N per μl blood | [mm²] |
| Erythrocytes | 7.5 | 4 × 10⁶ to 5 × 10⁶ | 1.3 × 10³ to 1.7 × 10³ |
| Leukocytes | 7 to 20 | 4 × 10³ to 9 × 10³ | 0.2 to 2.8 |
| Thrombocytes | 1.5 to 3 | 1.5 × 10⁵ to 3 × 10⁵ | 0.3 to 2.1 |

Additionally, because the solid constituents of blood barely differ from blood plasma in terms of their acoustic properties and because only a very few scatter processes arise overall, for backscattering processes in the blood consideration is generally only given to backscatterings on erythrocytes. It is furthermore assumed that red corpuscles are spherical scattering bodies that do not interact with one another. While both assumptions apply without any limitations to the typically-used blood mimicking fluids, they only apply partially to human blood: the biconcave discs are randomly oriented and change their orientation continuously in a turbulent flow. It can therefore be assumed that within a given spatial angle, the visible surface is always the same size, i.e., the scattering cross section is not direction-specific.

However, if one considers a laminar flow, as is the case in the large arteries and in artificial blood vessels, then the erythrocytes orient themselves in the flow in order to decrease their drag. Owing to the volume fraction of blood cells of up to 50% of the total blood volume, of which erythrocytes make up the majority, the erythrocytes are continuously colliding with each other. Because they minimize the distance to neighboring cells, their drag is reduced substantially. This aggregation of erythrocytes, also known as rouleau formation due to its characteristic resemblance to coin rolls, leads to a directional dependency of the scattering signal on the one hand and to a signal increase on the other.

Erythrocyte aggregation is not to be confused with thrombocyte aggregation, which is a preliminary stage of clotting. Owing to the considerably smaller dimensions, a change in the ultrasonic backscattering signal based on the thrombocytes alone is not visible.

Put simply, coagulation is based on the combining of red blood corpuscles with fibrin threads to create an insoluble network that can, for example, close a wound but also obstruct a blood vessel. Coagulation is therefore an effect that can have positive as well as negative consequences.

The invention is based on the surprising finding that the coagulation state can be safely detected as a characteristic of blood by ultrasonic measurements if not only changes in the distribution of blood constituents are measured by means of backscattering, but also if changes in the viscosity of the blood and/or of the bloodstream are additionally and/or simultaneously measured. The cross-linking of blood corpuscles with fibrin threads during clotting not only leads to significant elevation of the scattering cross section of the blood constituents, which can be measured by the ultrasonic backscattering, but also to a viscosity change that can be detected by means of elastography measurements.

In a particularly preferred embodiment, the ultrasonic monitoring of the fluid flow is performed by subjecting the fluid flow to time-harmonic mechanical and/or transient mechanical excitation and analyzing the ultrasonic backscattering signal in terms of the Doppler frequency shift and the elastography. From this it is possible to establish changes in the dynamic viscosity of blood as well as changes in the distribution of blood cells, which are ideally suited as markers for clotting states of blood.

Preference is given to exciting, in the fluid flow, low-frequency harmonic and/or non-harmonic waves, which are used to characterize the elastic properties of the fluid or fluid flow. The excitation of waves can be effected mechanically, pneumatically, and/or acoustically, as shown with examples in the following:
Mechanically: —Excitation by shaking or tapping
   Periodic deformation of the tube
   Exploitation of roller pump strokes for excitation
   Use of peristaltic pumps
Pneumatically: —Excitation by means of pneumatically controlled pressure cuffs
   Excitation by changing the pressure in the reservoir (Propagation of waves in the medium to the measurement point)
Acoustically: —Excitation with low-frequency transducers (loudspeakers)
   Coupling via air or suitable sound delay lines
   Excitation with an ultrasonic burst with a suitable PRF (Pulse repetition frequency)

In another embodiment, the method according to the invention is characterized in that a plurality of features of the blood state can be established instantaneously by means of the ultrasonic monitoring. With a “plurality of features of the blood state” is meant the physical parameters that can be measured by means of ultrasonic analysis, preferably ultrasonic backscattering, and that may be altered by the progressing blood coagulation. These parameters are preferably selected from the group comprising the sonic speed, the viscosity, the diameter of the scattering bodies, the standard deviation of the backscattering, the maximum deviation of the backscattering, the degree of regularity of the arrangement of scattering bodies, the turbulence and the velocity distribution of the scattering bodies. Among other things, the method according to the invention has the advantage that not only the progressing blood coagulation, but also the onset or beginning of the blood coagulation can be detected instantaneously. As parameters for detecting incipient clotting, particular preference is given to the viscosity and the scattering body diameter, the standard deviation of the backscattering, the maximum deviation of the backscattering, the degree of regularity of the arrangement of scattering bodies, the turbulence, and the velocity distribution of the scattering bodies. The scattering body diameter can vary significantly from the ca. 7 μm of an average erythrocyte to the several 10 to 100 μm of a microclot. Scattering body diameters, for example diameters of blood clots in the range of 10 to 150 μm, more preferably in the range 10 to 100 μm, just as preferably in the range of 15 to 80 μm, particularly preferably in the range of 15 to 50 μm, are preferably detectable with the method according to the invention.

As described above, the method according to the invention is based on the fact that the features of the fluid state, in particular of the blood state, established by means of the ultrasonic monitoring are further processed by means of a multi-criteria ultrasonic analysis. In a preferred embodiment, the method according to the invention is characterized in that the multi-criteria ultrasonic analysis of the fluid state is carried out by means of an analysis algorithm, which shall be described in the following.

The monitoring algorithm of the fluid state consists of a multi-layer model.

In level 1 (bottommost layer), the features of the ultrasonic analysis are displayed by backscattering, Doppler frequency shifting, and/or elastography. Their display can be linearly and non-linearly altered with the aid of display parameters.

In level 2, an application-oriented, statistical, numerical classification or regression is performed on the basis of the features from level 1. A distinction is made between a manual/mechanical (controlled) or automatic (uncontrolled) classification. The linkage of both methods is also possible. Whereas the classification searches for and allocates patterns in the features, the regression illustrates the correlation of a dependent variable (e.g., the fluid state) and one or a plurality of independent variables (e.g., viscosity and variance of the particles within the suspension) in a quantitative correlation. The display parameters of level 1 are optimized via machine learning online or offline, in order to display the features in an optimum fashion (minimization of errors) for analysis.

In level 3 (topmost layer), a state space model is formulated on the basis of the fluid states established in a time-discrete manner in level 2. Using a Bayesian minimum variance estimator for linear stochastic systems (Kalman, R. E., 1960, A new approach to linear filtering and prediction problems. Transaction of the ASME, Journal of Basic Engineering, pp. 35-45), the system state of the fluid can be optimally deduced (minimization of errors) from the results of level 2 that are subject to uncertainty. Accordingly, the state space model includes all available information, i.e., the knowledge of the physical process of the fluid and the dynamics of the measurement system, the underlying statistical processes of the system noise, the measurement error, and the uncertainty in the physical model, as well as the starting conditions.

The goals of this method are
a quick and an as accurate as possible estimation of the state of the fluid,
excellent resistance to interferences, whether in the individual physiological state of a patient or system-induced,
a quick reaction time to changes, regardless of whether they are intentional (e.g., change in the patient's medication) or unintentional (e.g., blood clotting),
a substantial reduction in the redundancy of the features present in level 1 to a few features for a high level of distinguishability of physical fluid states,
the online monitoring of the dynamics of a fluid.

In a particularly preferred embodiment, the analysis algorithm of the method according to the invention comprises the following signal processing steps:
a. Reception of an ultrasonic signal or a plurality of ultrasonic signals,
b. Extraction of features from the ultrasonic signal(s),
c. Pattern recognition,
d. Further processing, and
e. Assignment of a score, wherein the score defines the quality (probability) for a given classification result.

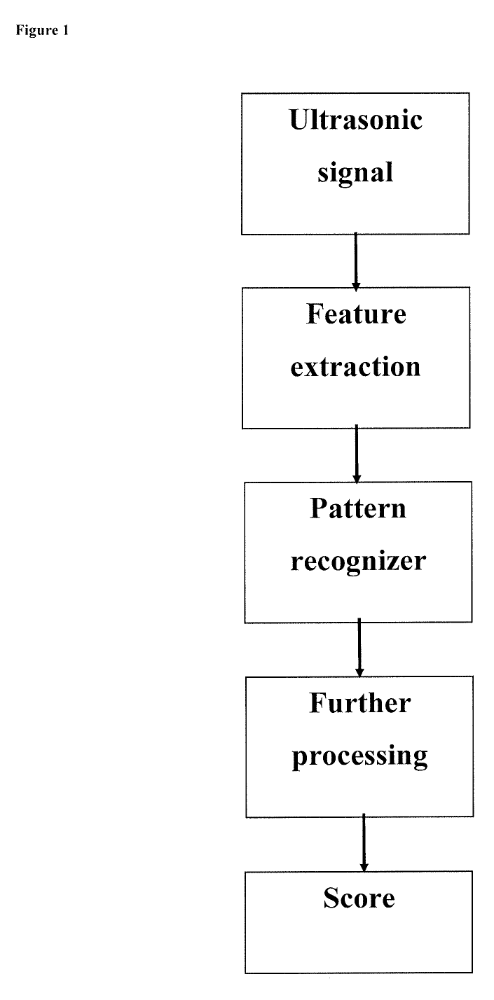

The embodiment presented here is based on the general structure of an analysis algorithm. The processing steps of a suitable analysis algorithm are illustrated in FIG. 1. Features describing changes in the coagulation as closely as possible are extracted from the ultrasonic signals. For this reason a fluid, preferably blood, is examined during controlled physical changes (e.g., harmonic motion excitations) in order to depict changes occurring among the particles, preferably among the blood cells, contained in the fluid. Next come the pattern recognizer and a further processing of the results in order to achieve an as conclusive as possible classification. Lastly, the “score” indicates the likely remaining error in the description of the state of the fluid, preferably of the blood, and thus represents the risk assessment of derivative procedures on the patient.

The pattern recognizer in the embodiment presented here is based on a serial application of principal constituent analysis (PCA) and linear discriminant analysis (LDA). This classification approach turns out to be equal in performance to the often-used support vector machine. However, it is better suited for discrimination problems with more than 2 classes (Li et al., 2003, Using discriminant analysis for multi-class classification. Proceedings of the Third IEEE International Conference on Data Mining, page 589. IEEE Computer Society.). With PCA, the signal information contained in the features can first be transformed into a compact representation. Although PCA is the ideal approach for this, it is not ideal in terms of discrimination between the classes. To this end, LDA is then used in order to separate the classes in an optimum manner by a coordinate transformation.

The method according to the invention, in particular the analysis algorithm according to the invention, is suitable for detecting criteria of the highest level of discrimination even in the “raw materials.” In another embodiment, the method according to the invention, in particular the analysis algorithm according to the invention, is based on a machine learning algorithm (a stochastic optimization of the imaging parameters of the features was carried out with a genetic algorithm), in which all signal processing steps, in particular the feature extraction, are optimized offline.

In another preferred embodiment, the analysis algorithm is adjusted online to the recognition problem via a continuous optimization. In a particularly preferred embodiment of the method according to the invention, the analysis algorithm additionally learns the individual state of the fluid, in particular the blood state, online as well. A further improvement in the robustness of the analysis logarithm is achievable with this optimization.

Using the method according to the invention, the analysis algorithm can detect changes in the state of the fluid, in particular the blood state, online. Using the analysis algorithm, the clotting state of blood or changes in the clotting state of blood can thus be detected online and with high reliability.

The advantage of the analysis algorithm for establishing clotting in blood described here over methods known to the prior art lies in the coupling of a multi-criteria extraction of features from the ultrasonic backscattering signal with a high-performance analysis of the fluid state. In particular the fluid flow, for example a flowing suspension such as a bloodstream, is subjected to time-harmonic mechanical and/or transient mechanical excitation and the ultrasonic backscattering signal is analyzed in terms of Doppler frequency shifting and elastography. From this it is possible to deduce the dynamic viscosity of blood as well as the change in the distribution of the blood cells, which are ideally suited as markers for blood clotting states. The analysis algorithm adapts to the database and isolates features exhibiting the highest distinguishability within the different clotting states. Large volumes of data on features can be effectively analyzed in this manner.

The method according to the invention furthermore has the advantage that the alterations of the clotting state of the blood detected online or rather instantaneously can be used for carrying out inventions. Examples of such interventions can include the immediate stopping of the extracorporeal circulation in, for example, a hemodialysis machine, or the systematic [unknown 1] addition of anticoagulants. Hence the method according to the invention in particular has the advantage of permitting a very quick reaction, for example within a few seconds, to changes in the clotting state of blood in an extracorporeal circuit and thus ensuring that no harm comes to the patient.

The method according to the present invention is also suitable for detecting errors in a fluid conduit means of a device. The fluid conduit means can be, for example, a tube system. The device is preferably a device for treating blood, such as a hemodialysis machine, a heart-lung machine, or an apheresis machine, for example.

The fact that the method according to the invention is a non-invasive method is particularly advantageous. Hence this method has the advantage that it is possible to dispense with performing invasive examinations (e.g., drawing blood) on the patient. Another advantage of the method according to the invention resides in the fact that the fluid state, in particular the blood state, can be measured continuously and over a long period.

The present invention also relates to a device for performing the method according to the invention. This device comprises at least one ultrasonic monitoring means and at least one signal analysis means. The device according to the invention is characterized in that the features of the fluid state, in particular of the blood state, established by means of the ultrasonic monitoring are processed by means of a multi-criteria analysis. Preference is given to use of the analysis algorithm described above (also see FIG. 1) in the device according to the invention for the multi-criteria ultrasonic analysis. With the processing of the features of the fluid state, in particular of the blood state, established by means of the ultrasonic monitoring, it is possible to measure alterations in the distribution of particles contained in the fluid, in particular blood constituents, and/or alterations in the viscosity of the fluid, in particular of blood, and assign pre-defined states to the fluid state, in particular to the blood state.

The device according to the invention has the advantage that changes in the state of a fluid, in particular in the coagulation state of blood, can be confirmed early, preferably from the beginning of the onset of the state change on.

With the device according to the invention it is also possible to detect at least one foreign structure and/or distinguish it from a second foreign structure in the fluid.

In the case of blood, the previously defined states are in particular clotting states. The primary aim of the detection of solid bodies in the blood is to detect clots.

Basically all measurement principles with which foreign structures, for example solid bodies and in particular blood clots, can be detected can be used for the ultrasonic monitoring. Preferred ultrasonic monitoring means according to the present invention are based on the measurement of backscattering, frequency shifting (Doppler), and/or viscosity (elastography). In a particularly preferred embodiment, an ultrasonic monitoring means based on the measurement of backscattering is employed in the device according to the invention.

The ultrasonic monitoring means integrated in the device according to the invention can use, for example, a measurement method based on ultrasonic backscattering for the particle characterization. The core idea of the design of such an ultrasonic monitoring means is the use of broadband transmission/reception transducers, which are optimally tuned to one another in respect of a necessary transmission output and a measurement window corresponding to the diameter of the fluid conduit means. In this manner the portion of sound scattered by the particles is detectable and analyzable in terms of the intensity, the runtime (which corresponds to the penetration depth), and the sound frequency. There are various possibilities for the arrangement of the ultrasound transducers. On the one hand, the direct backscattering can be measured by means of a single transmission/receiving transducer with a delay line. A standard reflection measurement is performed here. On the other hand, it is also possible to analyze the portions of scattered sound from defined angles. As a rule a separate pair of ultrasound transducers is used for this purpose.

However, it is also conceivable for the ultrasonic monitoring means integrated in the device according to the invention for the non-invasive measurement of foreign constituents in the fluid flow to be based on the ultrasonic Doppler effect method using ultrasound transmitters/receivers for the ultrasound reflected from the flowing blood and using Doppler frequency shifting between transmission and receiving frequency determining signal analysis means.

It is furthermore also conceivable for the ultrasonic monitoring means integrated in the device according to the invention for the non-invasive measurement of foreign constituents in the fluid flow to be based on the elastography method for determining the dynamic viscosity using ultrasound transmitters/receivers and a device for subjecting the fluid to time-harmonic mechanical and/or transient mechanical excitation.

In a particularly preferred embodiment, the ultrasonic monitoring means integrated in the device according to the invention for the non-invasive measurement of foreign constituents in the fluid flow is based on the elastography method for determining the dynamic viscosity using ultrasound transmitters/receivers and a device for subjecting the fluid to time-harmonic mechanical and/or transient mechanical excitation and additionally on the ultrasonic Doppler effect method using ultrasound transmitters/receivers for the ultrasound reflected from the flowing blood and using Doppler frequency shifting between transmission and receiving frequency determining signal analysis means.

The signal analysis means preferably comprises a standard processor and software for controlling the components and for signal processing and conversion. The signal analysis means is, for example, a control computer or a process computer, wherein the control computer or process computer can also be the control unit of a blood treatment device, for example a hemodialysis machine, a heart-lung machine, or an apheresis apparatus, said control unit being hooked up to the device according to the invention.

In particular, provision is made such that one or more solid bodies in the fluid, in particular blood clots, are detectable and are detected in the extracorporeal circuit.

In another preferred embodiment, by means of the signal analysis means the detection of at least one foreign structure in the fluid or fluid flow is recorded as a signal-triggering event, and in response the signal analysis means can emit a signal. Accordingly, a signal is preferably emitted by the signal analysis means when at least one blood clot is detected in the bloodstream of an extracorporeal circuit. The signal emitted by the signal analysis means is preferably relayed at regular intervals (for example at fixed intervals of a few milliseconds) to the control unit of the device and processed therein.

In another embodiment, the device according to the invention can have a receptacle into which a fluid conduit means, a cartridge, or a measurement channel can be inserted. The fluid conduit means is preferably a tube system or a part of a tube system, for example a tube system of the extracorporeal circuit of a hemodialysis machine, a heart-lung machine, or an apheresis apparatus. The cartridge can be, for example, a disposable cartridge such as those used in a hemodialysis machine for the arrangement of parts of an extracorporeal circuit. The device preferably has a measurement channel, in which the ultrasonic monitoring means can be arranged.

In a preferred embodiment, the device according to the invention can have an alarm means and/or be hooked up to at least one alarm means. This has the advantage that an alert to the presence of foreign structures detected in the fluid or fluid flow can be given with the alarm means. Preferably, an alert to the presence of solid bodies such as blood clots in the blood of an extracorporeal circuit can be given with the alarm means. The alarm means can be an acoustic (sound) and/or an optical (warning light or warning message on a computer screen) alarm means, for example. On the computer screen of a control unit that is hooked up to, for example, a hemodialysis machine, a heart-lung machine, or an apheresis apparatus, it is conceivable for a warning message to be given and an acoustic warning signal to be emitted simultaneously.

In another preferred embodiment, the device according to the invention comprises at least one protection means or is hooked up to at least one protection means. As described above, it is a particular advantage of the method according to the invention that changes in the state of a fluid flow, especially in the clotting state of the blood of an extracorporeal circuit, are detectable instantaneously. The device according to the invention, which has a protection means, has the advantage that it is possible to react immediately and without delay to alterations in the state of a fluid flow, for example in the clotting state of blood. For example, it is conceivble that the fluid flow can be stopped immediately after the detection of at least one foreign structure, in particular of at least one blood clot. It is thus possible to prevent any blood clots forming in an extracorporeal circuit of, for example, a hemodialysis machine or a cardiovascular machine from reaching the patient hooked up to the extracorporeal circuit. Immediately after the detection of a foreign structure, in particular a blood clot, a further task of the protective means can be to add means for dissolving these foreign structures into the extracorporeal circuit. Preferably, anticoagulants can be added to the extracorporeal circuit. Hence with the device according to the invention, very effective protection means are available for increasing patient safety and for preventing harm from coming to patients who have to undergo treatment with, for example, a hemodialysis machine or a cardiovascular machine or an apheresis apparatus.

In a particularly preferred embodiment, the device according to the invention is a component of a blood treatment device, in particular of a hemodialysis machine, a cardiovascular machine, or an apheresis apparatus.

The invention furthermore relates to the use of the device described herein for carrying out the method according to the invention. Preferably, the present invention also relates to the use of a device as described herein in a blood treatment device, in particular a hemodialysis machine, a heart-lung machine, or an apheresis apparatus.

In a particularly preferred embodiment of the present invention, the method described herein and/or the device described herein are used for the instantaneous and/or individual determination of the progression of the intrinsic and/or extrinsic blood coagulation in an extracorporeal circuit, particularly in a hemodialysis machine or a heart-lung machine or an apheresis apparatus.

The method according to the invention is not limited to the monitoring of the coagulation state of bloodstreams in extracorporeal circuits; rather, the method according to the invention and/or the device according to the invention can also be used in rapid testing systems, for example in POC (point of care) rapid systems, in outpatient and clinical practice, and in laboratory diagnostic devices, in outpatient and clinical practice.

The monitoring of the coagulation system typically takes place in a laboratory setting, in other words in vitro. Although the results are available after a few minutes, depending on the method, the time taken for drawing the blood, transport to the laboratory, and notification of the result must also be taken into account. Even state-of-the-art mobile devices, with which the time between drawing the blood and obtaining the result can be reduced to one minute, are limited to invasive measurement and are not suitable for monitoring coagulation parameters, e.g., during an operation.

In the past, the clotting characteristics of blood were determined by administering coagulation-activating substances that acted at different points of the coagulation cascade in order to monitor the action of the different clotting factors and/or the two pathways (extrinsic, intrinsic). The time until a defined clotting stage is reached, which is measured optically (with the naked eye or via transmission and/or scattering measurements with a sensor) or mechanically (e.g., ball coagulometer), only permits a statement regarding the probability of the onset of clotting in the patient, but not regarding the actual coagulation state in the blood.

The present procedure therefore represents a non-invasive method for determining the clotting state of blood in real time. In contrast to the biochemical methods used in the past for detecting hemostasis, with ultrasonic backscattering no statement can be made regarding the clotting factors, but one can be made regarding the actual clotting state in comparison to, say, normal blood. In the past this could only be estimated from empirical values.

The method according to the invention for monitoring a fluid state can in principle be used in all areas in which the state of fluids can change. For example, this applies to the monitoring of the mixing and/or separation state of all types of suspensions, dispersions, and emulsions. Examples of industrial applications of the method according to the invention include monitoring the state of suspensions and dispersions during the production of abrasives, paints, and varnishes.

The invention is characterized by the following features and subject matter in particular:

1. A method for establishing and/or monitoring the state of a fluid or fluid flow, wherein the fluid is monitored by means of ultrasound, characterized in that the features of the fluid state established by means of the ultrasonic monitoring are processed with a multi-criteria ultrasonic analysis, in particular with an analysis algorithm, by which a change in the distribution of particles contained in the fluid and/or changes in the viscosity of the fluid are measured and the fluid state is assigned to previously defined states.

2. The method according to subject matter 1 for establishing and/or monitoring the state of blood or of a bloodstream, in particular of an extracorporeal bloodstream, wherein the blood is monitored by means of ultrasound, characterized in that the features of the fluid state established by means of the ultrasonic monitoring are processed with a multi-criteria ultrasonic analysis, in particular with an analysis algorithm, by which a change in the distribution of constituents in the blood and/or changes in the viscosity of the blood are measured and the blood state is assigned to previously defined states, in particular to clotting states.

3. The method according to one of subject matters 1 or 2, characterized in that at least one first foreign structure, in particular a solid body such as a blood clot, can be detected and/or distinguished from at least one second foreign structure in the fluid.

4. The method according to at least one of the preceding subject matters, characterized in that the ultrasonic monitoring is based on the measurement of backscattering, frequency shifting (Doppler), and/or viscosity (elastography).

5. The method according to at least one of the preceding subject matters, characterized in that a plurality of features of the blood state can be established instantaneously by means of the ultrasonic monitoring.

6. The method according to at least one of the preceding subject matters, characterized in that the analysis algorithm comprises the following signal processing steps:
a. Reception of one ultrasonic signal or a plurality of ultrasonic signals,
b. Extraction of features from the ultrasonic signal(s),
c. Pattern recognition,
d. Further processing, and
e. Assignment of a score, wherein said score defines the quality (probability) for a given classification result.

7. The method according to at least one of the preceding subject matters, characterized in that all signal processing steps are optimized offline on the basis of a machine-learning algorithm.

8. The method according to at least one of the preceding subject matters, characterized in that the individual state of the fluid, in particular the blood state, is also learnable online by means of the analysis algorithm.

9. The method according to at least one of the preceding subject matters, characterized in that alterations in the fluid state, particularly in the blood state, are detectable online by means of the analysis algorithm.

10. The method according to at least one of the preceding subject matters, characterized in that with reference to the alterations detected online, the clotting state of blood is assignable to interventions such as the administration and/or concentration increase of an anticoagulant during hemodialysis, for example.

11. The method according to at least one of the preceding subject matters, characterized in that physical parameters that can change as a result of the progressing blood coagulation can be measured by means of ultrasonic backscattering.

12. The method according to at least one of the preceding subject matters, characterized in that physical parameters for detecting incipient blood coagulation can be measured by means of ultrasonic backscattering.

13. The method according to subject matter 10 or 11, characterized in that the physical parameters are selected from the group comprising the sonic velocity, the diameter of the scattering body, the standard deviation of the backscattering, the maximum deviation of the backscattering, the degree of regularity of the arrangement of the scattering bodies, the turbulence and velocity distribution of the scattering bodies, and the pressure in the tube system.

14. The method according to at least one of the preceding subject matters, characterized in that the aggregation of thrombocytes and/or erythrocytes is measurable.

15. The method according to at least one of the preceding subject matters, characterized in that the fluid flow, in particular the bloodstream, is subjected to time-harmonic mechanical excitation and the ultrasonic backscattering signal is analyzed in terms of the Doppler frequency shifting and the elastography.

16. The method according to at least one of the preceding subject matters, characterized in that errors are detectable in a fluid conduit means, preferably in a tube system of a device, in particular of a blood treatment device, preferably of a hemodialysis machine or heart-lung machine.

17. The method according to at least one of the preceding subject matters, characterized in that the method is non-invasive.

18. A device for carrying out the method according to at least one of the preceding subject matters, comprising at least one ultrasonic monitoring means and at least one signal analysis means, characterized in that the features of the fluid state, in particular of the blood state, established by means of the ultrasonic monitoring are processed with an analysis algorithm, by which alterations in the distribution of particles contained in the fluid, in particular blood constituents, and/or alterations in the viscosity of the fluid, in particular blood, are measurable and the fluid state, in particular the blood state, can be assigned to previously defined states, in particular clotting states.

19. The device according to subject matter 18, characterized in that at least one first foreign structure, in particular one or more solid bodies, in particular blood clots, are detectable and/or distinguishable from at least one second foreign structure in the fluid by means of the signal analysis means.

20. The device according to subject matter 18 or 19, characterized in that by means of the signal analysis means, alterations in the distribution of particles contained in the fluid, in particular of blood constituents, and/or alterations in the viscosity of the fluid, in particular blood, are recorded as a signal triggering event and a signal is emitted.

21. The device according to at least one of subject matters 18-20, characterized in that the device has a receptacle into which a fluid conduit means, preferably a tube system or part of a tube system or a cartridge, in particular a disposable cartridge, or a measurement channel can be inserted and/or that the device has a measurement channel.

22. The device according to at least one of subject matters 18-21, characterized in that the device has at least one alarm means and/or is hooked up to at least one alarm means, wherein by means of the alarm means, an alert can be given to changes in the distribution of particles contained in the fluid, in particular blood constituents, and/or to changes in the viscosity of the fluid, in particular blood.

23. The device according to at least one of the subject matters 18-22, characterized in that the device has at least one protection means and/or is hooked up to at least one protection means, wherein by means of the protection means the fluid flow is preferably stoppable and/or at least one means for correcting the state of the fluid, in particular the clotting state of blood, such as anticoagulants can be added to the fluid flow, in particular to the extracorporeal circuit.

24. The device according to at least one of subject matters 18-23, characterized in that the device is a component of a blood treatment device, in particular of a hemodialysis machine.

25. A use of a device according to at least one of subject matters 18 through 24 for carrying out the method according to at least one of subject matters 1 through 17 and/or in a blood treatment device, in particular a hemodialysis machine or a heart-lung machine.

26. A use of the method according to at least one of subject matters 1-17 or of the device according to at least one of subject matters 18-24 for the instantaneous and/or individual determination of the course of the intrinsic and/or extrinsic blood coagulation in an extracorporeal circuit, particularly in a hemodialysis machine or heart-lung machine.

27. The use of the method according to at least one of subject matters 1-17 in rapid testing systems such as POC (point of care) rapid systems; or in laboratory diagnostic devices, in outpatient and/or clinical practice.

In the following, further details of the invention will be explained more fully in the drawings and in an exemplary embodiment.

Shown are:

FIG. 1: the typical structure of the analysis algorithm according to the invention.

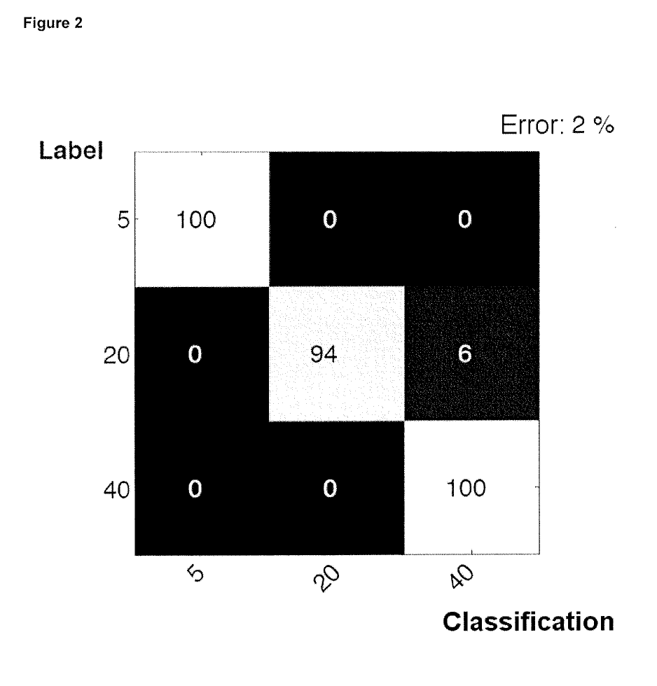

FIG. 2: the classification results from the differentiation of particle sizes in varying volume concentrations. The sizes are expressed in μm on the axes.

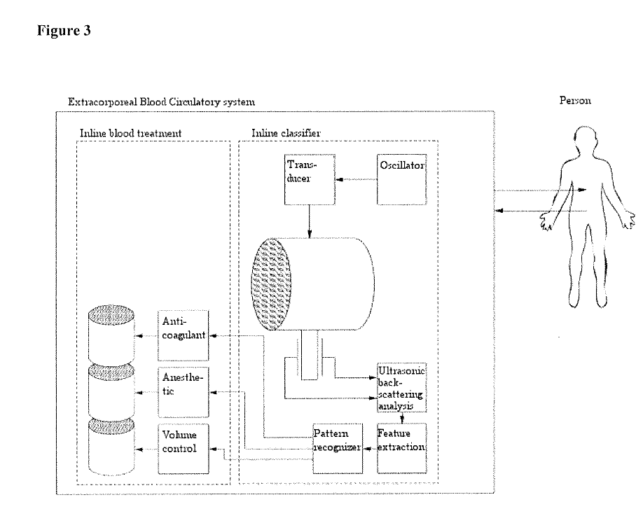

FIG. 3: the typical structure of extracorporeal blood circulation system with the corresponding ultrasonic monitoring means according to the present invention.

### EXAMPLE

The aim is to develop bases for the scattering theory of blood cell aggregation, impact of the coagulation cascade on the physical characteristics of the blood, and medically relevant parameters.

A first population of blood mimicking fluids (suspensions) with slightly varying volume concentrations at different mean particle sizes (5, 20 and 40 μm) was used for training an analysis algorithm. A second population with the same volume concentrations and particle sizes was then used for testing the quality of the analysis algorithm.

1. Technical design

An ultrasound device possessing a high signal-noise ratio was used for the experimental verification. The device was optimized for Doppler analysis by transmitting an ultrasonic burst (several oscillation periods). In addition, an analysis software permitting a high scanning speed of the pulse repetition frequency was implemented. This was required in order to satisfy Shannon's sampling theorem for the elastographic analysis. The classification was carried out with Matlab (The MathWorks Inc.).

The mechanical assembly was performed on a vibration couch with two electromechanical transducers. A holder for the cuvettes was placed on the two transducers. The suspension was made to vibrate by operating the transducers in counter-phase mode. The assembly comprised an analysis computer, units for supplying power to the stirrer and the ultrasound interface, as well as a cuvette that was mounted on a cuvette holder, i.e., on the transducers.

The plane-parallel walls made it possible to generate a complex network of modes, which interacted with the viscous properties of a suspension. For the Doppler measurements, a stirrer was immersed in the suspension and ultrasonic measurements were performed at different stirring speeds. For pure backscattering measurements, the suspension was stirred and measurements were taken with the stirrer operating at a very low rotational speed.

2. Preparation of a blood-like fluid

Human blood is not used directly in many areas of medical research, because among other things it does not store well. Because often only a few specific characteristics are used, which only in these characteristics behave like true blood. In addition to animal blood, partially and fully synthetic fluids, so-called blood-mimicking fluids (BMF), are also used. BMFs suitable for examinations to detect blood coagulation (hemostasis) by means of ultrasonic backscattering are used in this example.

BMF preliminary examinations for the backscattering method were performed on different

BMFs, which had to fulfill the following requirements:
different particle sizes,
suspensions, i.e., separable particle-solvent mixtures,
chemical and physical stability of the BMF.

BMFs were produced using various substances. The dependency of the BMF properties on the properties of the substances was investigated.

Materials: Two different substances were used to prepare BMFs: ORGASOL® (Arkema Inc.) and megaCRYL® (megadental GmbH).

TABLE 3
Properties of the substances used

| | megaCRYL ® | ORGASOL ® |
|---|---|---|
| Material | PMMA | PA12 |
| Diameter | 10-60 μm | 5 μm; 20 μm; 40 μm |
| Density | 1.16 g/cm³ | 1.03 g/cm³ |
| Original intended use | Cold-cure denture | Paint industry additive |
| polymerizer | | |

PMMA Polymethyl methacrylate
PA12 Polyamide 12

Both substances exhibited different behavior when stirred and in terms of the storability of the suspensions, as shown in Table 4.

TABLE 4
Properties of the BMFs produced

| | megaCRYL ® | ORGASOL ® | ORGASOL ® | ORGASOL ® |
|---|---|---|---|---|
| Scattering body | 10-60 μm | 5 μm | 20 μm | 40 μm |
| diameter | | | | |
| Dispersity | polydisperse | monodisperse | monodisperse | monodisperse |
| Suspendability in | very good | poor | good | good |
| water | | | | |
| Viscosity | yes | no | no | no |
| influenced by | | | | |
| concentration? | | | | |
| Resuspendability | moderate | moderate | good | good |
| Chem. stability | Polymerization | 1) | 1) | 1) |
| Phys. stability | 1) | 1) | 1) | 1) |
| Biol. stability | 2) | 2) | 2) | 2) |
| Highest | 33 M. % | 25 M. % | 18 M. % | 18 M. % |
| concentration | | | | |
| produced | | | | |

1) no change observed thus far, i.e., the suspensions remained unchanged for months.
2) the suspensions do not contain any biological components, and
3) maximum possible concentration with additives used thus far.

Viscosity Measurement

A dependency of the viscosity on the concentration was established for the megaCRYL® suspensions. The viscosity of the ORGASOL® suspensions is similar to that of water.

Database

For verifying the classification method, the suspensions in Table 5 were created. As in the eventual dialysis machine application, a reliable detection of the particle diameter of individual blood cells and their microaggregates should be possible in spite of a varying volume concentration (hematocrit).

TABLE 5
Database for verifying the classification of suspensions differing
in terms of both particle diameter and particle number.

| Suspension (Set/Diameter) | Percent by volume |
|---|---|
| 1/5 | 10.1 ± 0.23 |
| 1/20 | 9.8 ± 0.24 |
| 1/40 | 9.3 ± 0.25 |
| 2/5 | 11.7 ± 0.24 |
| 2/20 | 11.7 ± 0.24 |
| 2/40 | 11.7 ± 0.24 |
| 3/5 | 13.8 ± 0.25 |
| 3/20 | 13.8 ± 0.25 |
| 3/40 | 13.6 ± 0.24 |
| 4/5 | 14.4 ± 0.25 |
| 4/20 | 14.6 ± 0.25 |
| 4/40 | 14.5 ± 0.25 |
| 5/5 | 15.7 ± 0.25 |
| 5/20 | 15.8 ± 0.26 |
| 5/40 | 15.8 ± 0.26 |
| 6/5 | 17.6 ± 0.26 |
| 6/20 | 17.8 ± 0.26 |
| 6/40 | 17.5 ± 0.26 |

3. Performance of the measurements

The measurements were performed using the ft2232 (GAMPT) software with the following settings: prt 1999 (plus-repetition rate), data num. 2048 (number of data points of the A-scan), delay 5 (start of the recording of data points), gain 50 (analog amplification in dB re 1V), transmitter 30 (analog amplification transducer dB re 1 V), filter 1 100 (no filtering), filter 2 100 (no filtering), spl 4000 (recording 4000 A-scan lines), fs 50 MHz (scanning frequency), burst f 3,125 Mhz (signal frequency), TGC max. (full digital amplification) in the measurement range using a panametrics transducer with a center frequency of 3.5 MHz.

From the measurements of sets 4 to 6, the features backscattering, Doppler, and elastography were determined on samples taken at different times, over a recording time of 0.6 seconds in each case. Accordingly, there were 9 suspensions×7 excitation forms (stirring speed, etc.)×6 different time samples for the training. The measurements and feature extractions of the 7 excitation forms were arranged in a vector, which ultimately resulted in 54 samples. The same number was used for the evaluation of the analysis algorithm. Both populations are temporally independent of one another. The analysis of backscattering, Doppler, and elastography was performed for each measurement, regardless of the excitation form. The depth range was limited to 300 data points in the backscattering range in order to avoid effects due to different fill levels in the cuvette. Each sample resulted in a vector of 7 excitation types×1068 features in each case. After they were calculated, all features were logarithmized (base e).

The seven excitation forms comprised supplying the stirrer with 0.7, 1, 1.5, and 2V (Hewlett Packard E3611A, at full amperage) of power as well as harmonic excitation with 5, 8, and 15 Hz (tamp TSA 1400 amplifier at 50% output, PC sound card type: Realtek Audio High Definition sound card, maximum amplification, Matlab with an amplitude factor of 0.5), with the stirrer turned on. Before each measurement, the suspension was briefly stirred in order to avoid sedimentation. The stirring speed at 0.7 V corresponds to a stationary state of the suspension per A-scan, which was a required criterion for the backscattering analysis performed here.

The evaluation results of the trained analysis algorithm are presented in FIG. 2 in the form of a so-called confusion matrix. The ordinate gives the actual label, in this case the diameter. The abscissa designates the classification results. The results are expressed as percents. A perfect classification would lead to a principal diagonal with values of 100% in each case. A very high classification quality with an error of 2% was attained in actuality.

The results of the described measurement verify the validity of the approach presented here of the multi-criteria examination of fluids for establishing particle size differences with varying volume concentrations far below the resolution of the ultrasonic signal (the wavelength of the ultrasonic signal used in water was 480 μm).

1. A method for establishing and/or monitoring the state of an extracorporeal fluid or of an extracorporeal fluid flow, wherein the fluid is monitored by means of ultrasound, wherein the extracorporeal fluid or the extracorporeal fluid flow is subjected to time-harmonic mechanical and/or transient mechanical excitation and produced ultrasonic backscattering signal is analyzed in terms of Doppler frequency shifting and elastography, features of the state of the extracorporeal fluid established by means of ultrasonic monitoring are processed with a multi-criteria ultrasonic analysis, in particular an analysis algorithm, by which a change in the distribution of particles contained in the fluid and changes in the viscosity of the fluid are measured by means of ultrasound and the fluid state is assigned to previously defined states.

2. The method of claim 1 wherein the extracorporeal fluid is blood and is monitored by means of ultrasound, wherein extracorporeal bloodstream is subjected to time-harmonic mechanical and/or transient mechanical excitation and the ultrasonic backscattering signal is analyzed in terms of Doppler frequency shifting and elastography, the features of the state of the extracorporeal blood established by means of ultrasonic monitoring are processed with a multi-criteria ultrasonic analysis, in particular an analysis algorithm, by which a change in the distribution of constituents in the blood and changes in the viscosity of the blood are measured by means of ultrasound and the blood state is assigned to previously defined states, in particular clotting states.

3. The method of claim 1, wherein at least one first foreign structure, in particular a solid body such as a blood clot, is detectable and/or distinguishable from at least one second solid/liquid/gaseous foreign structure, e.g., air bubbles, in the extracorporeal fluid.

4. The method of claim 1, wherein a plurality of features of the extracorporeal fluid state are established instantaneously by means of ultrasonic monitoring.

5. The method of claim 1, wherein the analysis algorithm comprises the following signal processing steps:
a. Reception of an ultrasonic signal or a plurality of ultrasonic signals,
b. Extraction of features from the ultrasonic signal(s),
c. Pattern recognition,
d. Further processing, and
e. Assignment of a score, wherein said score defines the quality (probability) for a given classification result.

6. The method of claim 1, wherein all signal processing steps are optimized offline on the basis of a machine learning algorithm.

7. The method of claim 1, wherein the individual state of the extracorporeal fluid, in particular the blood state, is in addition learnable online by means of the analysis algorithm.

8. The method of claim 1, wherein the extracorporeal fluid is blood and the coagulation state of the blood is assignable, with reference to online-detected changes, to interventions such as the administration and/or concentration increase of an anticoagulant during hemodialysis.

9. The method of claim 1, wherein the extracorporeal fluid is blood and physical parameters that are subject to change by progressing blood coagulation are measured by ultrasonic backscattering.

10. The method of claim 9, wherein physical parameters for detecting incipient blood coagulation are measured by ultrasonic backscattering.

11. The method of claim 9, wherein the physical parameters are selected from the group consisting of sonic speed, diameter of the scattering body, the standard deviation of the backscattering, the maximum deviation of the backscattering, the degree of regularity of the arrangement of the scattering bodies, the turbulence and the velocity distribution of the scattering bodies, the pressure in the tube system.

12. The method of claim 9, wherein the aggregation of thrombocytes and/or erythrocytes is measured.

13. The method of claim 1, wherein errors are detected in an extracorporeal fluid conduit.

14. A device for monitoring state of an extracorporeal fluid flow comprising at least one ultrasonic monitoring means and at least one signal analysis means, wherein the extracorporeal fluid flow, in particular the extracorporeal bloodstream, is subjected to time-harmonic mechanical and/or transient mechanical excitation and the ultrasonic backscattering signal is analyzed in terms of Doppler frequency shifting and elastography, the features of the state of the extracorporeal fluid, particularly of the state of extracorporeal blood, established by means of the ultrasonic monitoring are processed with an analysis algorithm, by which changes in the distribution of particles, in particular blood constituents, contained in the extracorporeal fluid and changes in the viscosity of the extracorporeal fluid, in particular of extracorporeal blood, are measurable by means of ultrasound and the state of the extracorporeal fluid, in particular of the extracorporeal blood, is assignable to previously defined states, in particular to coagulation states.

15. The device of claim 14, wherein the signal analysis means detects at least one first foreign structure, in particular one or a plurality of solid bodies, in particular blood clots, and distinguishes the first foreign structure from at least one second foreign structure in the extracorporeal fluid.

16. The device of claim 14, wherein the signal analysis means detects changes in the distribution of particles, in particular blood constituents, contained in the extracorporeal fluid and changes in the viscosity of the extracorporeal fluid, in particular extracorporeal blood, records signal-triggering events, and emits a signal.

17. The device of claim 14, wherein the device has a receptacle adapted to receive a fluid conduit means, preferably a tube system or a portion of a tube system or a cartridge, in particular a disposable cartridge, or a measurement channel.

18. The device of claim 14, wherein the device has at least one alarm means and/or is hooked up to at least one alarm means for alerting changes in the distribution of particles, in particular blood constituents, contained in the extracorporeal fluid, and to changes in the viscosity of the extracorporeal fluid, in particular of extracorporeal blood.

19. The device of claim 14, wherein the device has at least one protection means and/or is hooked up to at least one protection means for stopping extracorporeal fluid flow and/or at least one means for correcting the state of the extracorporeal fluid, in particular the clotting state of extracorporeal blood by addition of anticoagulants.

20-22. (canceled)

23. A device for monitoring state of an extracorporeal fluid which comprises
a conduit for extracorporeal fluid;
an ultrasonic transducer operably associated with the conduit;
an ultrasonic back scattering detector operably associated with the conduit; and
a multi-criteria signal analyzer operably associated with the ultrasonic back scattering detector.
